(19) United States
(12) Patent Application Publication
(10) Pub. No.: US 2008/0076142 A1
(43) Pub. Date: Mar. 27, 2008

(54) Method for Spatially High-Resolution Investigation of a Structure, Marked With a Fluorescing Substance, of a Specimen

(75) Inventors: Werner KNEBEL, Kronau (DE); Jan Schroeder, Mannheim (DE)

Correspondence Address: CROWELL & MORING LLP;INTELLECTUAL PROPERTY GROUP, P.O. BOX 14300, WASHINGTON, DC, 20044-4300, US

(73) Assignee: Leica Microsystems CMS GmbH, Wetzlar (DE)

(21) Appl. No.: 11/860,011

(22) Filed: Sep. 24, 2007

(30) Foreign Application Priority Data
Sep. 25, 2006 (DE) 10 2006 045 607.6

(51) Int. Cl. C12Q 1/26; C12Q 1/34

(52) U.S. Cl. 435/18; 435/25

(57) ABSTRACT

A method for spatially high-resolution investigation of a structure, marked with a fluorescing substance, of a specimen, the substance being capable of being repeatedly converted from a first state into a second state, the first and second states differing from one another in terms of at least one optical property, encompasses the following steps. Firstly, bring the substance, in a specimen region to be sensed, into the first state; and inducing the second state by way of an optical signal, spatially delimited subregions within the specimen region to be sensed being blanked out in controlled fashion. A protein—target protein—in living cells is used as a structure that is marked with the fluorescing substance, by the fact that a ligand complex encompassing the fluorescing substance is bound to an enzyme via an enzymatic reaction in the cell, the enzyme being expressed as a fusion protein together with the target protein to be investigated.

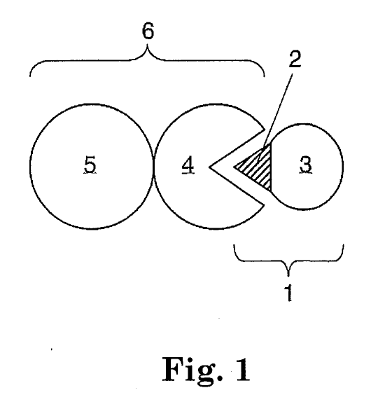

### BACKGROUND AND SUMMARY OF THE INVENTION

This application claims the priority of German Application No. 10 2006 045 607.6, filed Sep. 25, 2006, the disclosure of which is expressly incorporated by reference herein.

The invention relates to a method for spatially high-resolution investigation of a structure, marked with a fluorescing substance, of a specimen, the substance being capable of being repeatedly converted from a first state into a second state. The first and second states differ from one another in terms of at least one optical property. Such a method includes the steps of: firstly bringing the substance, in a specimen region to be sensed, into the first state; and inducing the second state by way of an optical signal, spatially delimited subregions within the specimen region to be sensed being blanked out in controlled fashion.

The invention furthermore relates to a method for spatially high-resolution investigation of a structure, marked with a fluorescing substance, of a specimen, the substance being capable of being converted from a first state into a second fluorescing state, and a spatially highly resolved image of the structure being prepared by irradiating the specimen with a predefinable small quantity of excitation light so that a small percentage of the fluorescent molecules transitions into the second state. The emitted light resulting from spontaneous decay of the individual excited fluorescent molecules in the second state is detected by a detection device, and the center point of the emitted light is calculated for each of the fluorescent molecules with the aid of suitable statistical methods. The fluorescent molecules in the second state are returned to the first state and/or bleached. The steps of irradiation, detection, center-point calculation, and returning to the first state and/or bleaching all molecules in the second state are repeated a large number of times, for a different subset of fluorescent molecules in each case, and an overall image is prepared from the individual images thus prepared.

Methods of the above-mentioned type have been known for some time from practical use. Microscopy methods that may be mentioned at this juncture merely by way of example are STED (stimulated emission depletion), RESOLFT (reversible saturable optical fluorescence transitions), GSD (ground state depletion), fluorescence upconversion, and PALM (photoactivated localization microscopy). With these imaging optical methods it is possible to achieve spatial resolutions beyond the theoretical limit, which according to Abbe's law is determined by the diffraction limit as a function of the wavelength of the illuminating light that is used.

In the context of STED microscopy, a substance that can be brought by light into an excited state, and that can be abruptly deexcited from this excited state, is made available in the specimen to be investigated. The substances of this kind that are very predominantly used in STED microscopy are fluorescent dyes. In general, the substance is first converted into the excited state with short-wavelength light, e.g. a green laser pulse. The substance is then deexcited in controlled fashion, in a peripheral focus region of the excitation, by use of a long-wavelength (e.g. red) laser pulse. In order to achieve deexcitation of the substance exclusively in the peripheral focus region, the deexcitation point function is specially shaped. Phase filters, which are located in the beam path of the long-wavelength laser beam and modify the wave front of the deexcitation light beam in positionally dependent fashion, are generally used for this purpose. It is critical that the transition from the excited to the deexcited state induced by the deexcitation light beam in the peripheral region take place in saturated fashion, i.e. completely, so that the substance remains in the excited state only in a (in principle, arbitrarily) small central region. Emission of fluorescent light from the peripheral region of the diffraction-limited excitation spot is thus prevented by the deexcitation light pulse. The detected fluorescent light therefore derives from a narrowly defined specimen region whose diameter, because of the saturation of the deexcitation, can be substantially smaller than permitted by Abbe's law.

In STED microscopy, dyes are generally used as a fluorescing substance to mark the structure to be investigated; in addition to a high quantum yield, these possess the further advantage that they do not bleach as quickly as standard fluorescent proteins, e.g. the known GFP (green fluorescent protein). This low bleaching tendency is very particularly important for STED, since in this process the specimen must be acted upon by high illumination intensities for saturated deexcitation of the fluorescing state.

It is disadvantageous in this connection that the fluorescent dyes under discussion here are organic molecules that lack suitable chemical groups for binding to the target proteins being investigated. The result of this circumstance is that in STED, a marking method mediated by immunofluorescence is used. For this, the cells to be investigated must be incubated with a suitable antibody that specifically recognizes the target protein. It is problematic in this context that marking via antibodies is not possible in living cells. In other words, in vivo marking is precluded, so that a spatially high-resolution investigation of physiological reactions proceeding within a living cell is not possible.

The object on which the present invention is based is now to configure and further develop methods of the above-mentioned type for spatially high-resolution investigation of a structure, marked with a fluorescing substance, of a specimen, so that utilization on the cell-biology level becomes possible and so that physiological processes within living cells can be imaged at high resolution.

This and other objects are achieved according to the present invention by a method for spatially high-resolution investigation of a structure, marked with a fluorescing substance, of a specimen, the substance being capable of being repeatedly converted from a first state into a second state, the first and second states differing from one another in terms of at least one optical property. The method encompasses the acts of: firstly bringing the substance, in a specimen region to be sensed, into the first state; and inducing the second state by way of an optical signal, spatially delimited subregions within the specimen region to be sensed being blanked out in controlled fashion, wherein a protein—target protein—in living cells is used as a structure that is marked with the fluorescing substance, by the fact that a ligand complex encompassing the fluorescing substance is bound to an enzyme via an enzymatic reaction in the cell, the enzyme being expressed as a fusion protein together with the target protein to be investigated.

What has been recognized according to the present invention is, firstly, that high-resolution investigation of physiological, cell-biology, and developmental-biology processes requires efficient in vivo marking, and that marking via antibodies is consequently precluded. According to the present invention, a ligand complex is made available that encompasses the fluorescing substance and that the cell binds to an enzyme via an enzymatic reaction. The enzyme is expressed in the cell as a fusion protein together with the target protein, since the gene sequence of the enzyme has previously been introduced into the genome of the cell.

Provision is made, advantageously, for the ligand complex to include a reactive linker, the ligand complex being covalently bound to the enzyme via the linker. For this purpose, the cells can be incubated with the ligand complex, and ligand complexes that have not yet entered into a bond with the enzyme after a predefinable incubation time can be washed out.

In the context of a particularly preferred embodiment, a genetically modified hydrolase protein, in which the catalytic base has been replaced with a phenylalanine radical, is used as an enzyme. Because the enzyme's ability to hydrolyze the ester intermediate is switched off by this exchange, the enzyme becomes catalytically inactive so that a stable covalent bond can be formed.

In the interest of a large variety of possible applications, provision can be made that the enzymatic reaction by which the ligand complex binds to the enzyme is a halogenation. The ligand complexes available in this case, which include a reactive linker having a corresponding chloroalkane, can penetrate the cell membrane and in general have no toxic effect on the cells. A reduction or an oxidation are also contemplated as alternative enzymatic reactions to a halogenation reaction.

The fluorescing substance that, in addition to the reactive linker, constitutes the second module of the ligand complex as a functional reporter can be a synthetic dye, no limits being set in principle regarding the nature of the dye. The fluorescent dye can be, for example, TMR, a dye having a high quantum yield, high photostability, and a low triplet population, or a functional reporter that is available with different excitation and emission wavelengths.

It is thus possible, for example, to use fluorescent dyes in which the first and the second state are states that fluoresce at different emission wavelengths. Alternatively, it is contemplated for the first state to be a fluorescing state, and the second state to be a non-fluorescing state.

For preparation of a spatially highly resolved image of the target proteins, it is also possible to use as a fluorescing substance switchable fluorescent dyes that can be switched between the fluorescing first state and the non-fluorescing second state by irradiation with light, by chemical reactions, by heat, or in another fashion. The first state can firstly be established locally within the specimen to be investigated. The second state is then switched on in saturated fashion in a peripheral focus region so that fluorescent molecules remain in the first state only in an (arbitrarily) small focus region. The emitted light proceeding from these fluorescent molecules as a consequence of spontaneous decay is detected by a detection device. These methods, in which the diffraction limit is overcome by way of a reversible saturated optical transition, are referred to generally as RESOLFT methods. A special case is represented by the STED method, in which a fluorescing excitation state is deexcited in saturated fashion, by irradiation with light at a suitable deexcitation wavelength, in a peripheral focus region of the excitation.

In the interest of a further improvement in information recovery, pulse-chase experiments for multiple-color applications can be performed in the context of the RESOLFT or STED methods. For example, the fusion proteins made up of an enzyme and a target protein bound thereto can be marked at time t=0 with “green” dye, at time t=1 with “yellow,” and at time t=2 with “red” fluorescent dye. This creates the capability, for example, of analyzing membrane trafficking in time and space.

As an alternative to the RESOLFT or STED method described, the aforesaid object is furthermore achieved by a method for spatially high-resolution investigation of specimens. In this method, a spatially highly resolved image of the target proteins is prepared by way of a statistical method. For this, the specimen is irradiated with a predefinable small quantity of excitation light so that only a small percentage of the fluorescent molecules coupled to the target proteins transition into the second, fluorescing state. In a next step, the emitted light resulting from spontaneous decay of the individual excited fluorescent molecules in the second state is detected by a detection device. The quantity of irradiated excitation light is selected so that the detected light of the individual excited fluorescent molecules is detectable in a manner spatially separated from one another. This creates the possibility of calculating, with the aid of suitable statistical methods, the center point of the emitted light for each excited fluorescent molecule. The steps of irradiation, detection, center point calculation, and returning to the first state or bleaching all molecules in the second state are repeated many times in chronological succession, a different subset of fluorescent molecules being excited in each cycle. The procedure can be repeated, for example, on the order of 10,000 times, and an overall image can be prepared from the individual images respectively prepared in this fashion. This procedure, known per se, is generally referred to as PALM (photoactivated localization microscopy).

There are various ways of advantageously embodying and refining the teaching of the present invention. The reader is referred, for that purpose, on the one hand to the dependent claims, and on the other hand to the explanation below of a preferred exemplifying embodiment of the invention with reference to the drawing. In conjunction with the explanation of the preferred exemplifying embodiment of the invention with reference to the drawing, an explanation is also given of generally preferred embodiments and refinements of the teaching.

### BRIEF DESCRIPTION OF THE DRAWING

The single FIGURE schematically depicts an exemplifying embodiment of a bond between a target protein and a fluorescing substance that can be used in the method according to the present invention for spatially high-resolution investigation.

### DETAILED DESCRIPTION OF THE DRAWING

The FIGURE schematically shows a ligand complex 1 that encompasses a reactive linker 2 as well as a functional reporter in the form of a fluorescent dye 3. In the exemplifying embodiment depicted in the FIGURE, fluorescent dye 3 is concretely an “ATTO dye” that is manufactured for STED by the ATTO TEC company, for example under the product designation ATTO 647 N.

Ligand complex 1 is bound to an enzyme 4 by enzymatic halogenation. Enzyme 4 is expressed as a fusion protein 6 together with a target protein 5 to be investigated. Enzyme 4 depicted in the exemplifying embodiment according to the FIGURE is, concretely, a protein marketed under the trade name HaloTag™ by the Promega company. This protein is derived from a prokaryotic hydrolase and can be used to generate an N-terminal or C-terminal fusion that can be expressed in a plurality of cell types.

In conclusion, let it be emphasized that the exemplifying embodiment described above serves merely for discussion of the teaching claimed, but does not limit the latter to the exemplifying embodiment.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for spatially high-resolution investigation of a structure, marked with a fluorescing substance, of a specimen, the substance being capable of being repeatedly converted from a first state into a second state, the first and second states differing from one another in terms of at least one optical property, the method comprising the acts of:
bringing the substance, in a specimen region to be sensed, into a first state;
inducing the second state by way of an optical signal, spatially delimited subregions within the specimen region to be sensed being blanked out in a controlled fashion; and
wherein a target protein in living cells is used as the structure marked with the fluorescing substance, in that a ligand complex encompassing the fluorescing substance is bound to an enzyme via an enzymatic reaction in the cell, the enzyme being expressed as a fusion protein together with the target protein being investigated.

2. The method according to claim 1, wherein the ligand complex is covalently bound to the enzyme via a reactive linker.

3. The method according to claim 1, wherein a genetically modified hydrolase protein, in which a catalytic base has been replaced with a phenylalanine radical, is used as the enzyme.

4. The method according to claim 2, wherein a genetically modified hydrolase protein, in which a catalytic base has been replaced with a phenylalanine radical, is used as the enzyme.

5. The method according to claim 1, wherein the enzymatic reaction is a halogenation.

6. The method according to claim 2, wherein the enzymatic reaction is a halogenation.

7. The method according to claim 4, wherein the enzymatic reaction is a halogenation.

8. The method according to claim 1, wherein the enzymatic reaction is a reduction or an oxidation.

9. The method according to claim 2, wherein the enzymatic reaction is a reduction or an oxidation.

10. The method according to claim 4, wherein the enzymatic reaction is a reduction or an oxidation.

11. The method according to claim 1, wherein the fluorescing substance is a synthetic fluorescent dye.

12. The method according to claim 11, wherein the fluorescent dye is a dye having at least one of: a high quantum yield, a high photostability, and a low triplet population.

13. The method according to claim 1, wherein the first and the second state are states that fluoresce at different emission wavelengths.

14. The method according to claim 1, wherein the first state is a fluorescing state, and the second state is a non-fluorescing state.

15. The method according to claim 14, wherein a spatially highly resolved image of the target proteins is prepared:
by illuminating the specimen, for local generation of the fluorescing first state, with light at a wavelength of the excitation spectrum of the fluorescing substance;
the specimen being illuminated with light of a suitable deexcitation wavelength for saturated generation of the non-fluorescing second state in a peripheral focus region of the excitation; and
the emitted light proceeding from the specimen, said light resulting from spontaneous decay of molecules remaining in the first state in a spatially narrowly limited subregion, being detected by a detection device.

16. The method according to claim 15, wherein pulse-chase experiments using differently colored fluorescing substances are carried out to ascertain the time dependence of physiological processes.

17. A method for spatially high-resolution investigation of a structure, marked with a fluorescing substance, of a specimen, the substance being capable of being converted from a first state into a second fluorescing state, and a spatially highly resolved image of the structure being prepared, the method comprising the acts of:
irradiating the specimen with a predefinable small quantity of excitation light so that a small percentage of the fluorescent molecules transitions into the second state;
detecting emitted light resulting from spontaneous decay of the individual excited fluorescent molecules in the second state via a detection device;
calculating a center point of the emitted light for each of said fluorescent molecules with the aid of suitable statistical methods;
at least one of returning the fluorescent molecules in the second state to the first state, and bleaching the fluorescent molecules;
repeating the acts of irradiating, detecting, center-point calculating, and returning to the first state and/or bleaching a large number of times, for a different subset of fluorescent molecules in each case; and
preparing an overall image from the individual images thus prepared, wherein a target protein in living cells is used as the structure marked with the fluorescing substance, in that a ligand complex encompassing the fluorescing substance is bound to an enzyme via an enzymatic reaction in the cell, the enzyme being expressed as a fusion protein together with the target protein to be investigated.

18. A method for spatially high-resolution investigation of a target protein in living cells marked with a fluorescing substance, the substance being capable of being repeatedly converted from a first state into a second state, the first and second states differing from one another in terms of at least one optical property, the method comprising the acts of:
binding a ligand complex encompassing the fluorescing substance to an enzyme via an enzymatic reaction in the living cell, wherein the enzyme is expressed a fusion protein together with the target protein to be investigated;
bringing the fluorescing substance, in a region of the target protein to be sensed, into a first state; and
inducing the second state by way of an optical signal, spatially delimited subregions within the region of the target protein to be sensed being blanked out in controlled fashion.

19. The method according to claim 18, wherein the binding act further comprises the act of covalently binding the ligand complex to the enzyme via a reactive linker.
